(19) United States
(12) Patent Application Publication
(10) Pub. No.: US 2016/0243184 A1
(43) Pub. Date: Aug. 25, 2016

(54) METHODS FOR NATURAL RELUBRICATION FOR VAGINAL ATROPHY

(71) Applicants: Vanessa Kaye Beetham, Carlsbad, CA (US); Dianne Lynn Harsch, San Diego, CA (US)

(72) Inventors: Vanessa Kaye Beetham, Carlsbad, CA (US); Dianne Lynn Harsch, San Diego, CA (US)

(21) Appl. No.: 14/706,267

(22) Filed: May 7, 2015

Related U.S. Application Data
(60) Provisional application No. 62/120,368, filed on Feb. 24, 2015.

(51) Int. Cl. A61K 36/889; A61L 15/44

(52) U.S. Cl. CPC A61K 36/889; A61L 15/44; A61L 2300/452; A61L 2300/30

(57) ABSTRACT

The invention provides a new option for women suffering from vaginal atrophy caused by the declining production of estrogen.

It provides methods using coconut oil as a natural lubricant/moisturizer for women who experience painful burning during intercourse due to dryness.

### BACKGROUND OF THE INVENTION

The following discussion of the background of the invention is merely provided to aid the reader in understanding the invention and is not admitted to describe or constitute prior art to the present invention.

A common occurrence in women is known as vaginal atrophy. It is thinning, drying and inflammation of the vaginal walls caused by the declining production of estrogen. This can occur at any age, but is most often in peri-menopausal, menopausal and post-menopausal women. It is also observed in any affliction that reduces estrogen such as during breastfeeding, surgical menopause, pelvic radiation or chemotherapy and breast cancer hormonal treatment.

Currently, vaginal atrophy is treated by topical application of lubricants known as vaginal moisturizer. Some well-known non-hormone containing products are Vagisal® or Replens®, that contain complex ingredients that are often messy and use synthetic polymers.

Women are looking for natural remedies that provide easy application, are mess-free, convenient and fit with their daily lifestyle.

### SUMMARY OF THE INVENTION

It is an object of the present invention to provide methods for producing a natural lubricant/moisturizer for women who experience the painful burning sensation during intercourse due to vaginal atrophy.

Refined or unrefined Coconut Oil infused Tampon, Suppository/aliquot, Sponge or Douche Packet delivered to the vagina with or without applicator.

### DETAILED DESCRIPTION OF THE INVENTION

As described herein, we provide details of the method to alleviate vaginal atrophy. As well as a possible therapeutic method to treat vaginal yeast infection. Vaginal atrophy can occur in any organism where estrogen is limited. In essence, we see possible veterinarian applications as well as the most preferred embodiment of the method with women.

Coconut oil is extracted from the matured coconuts harvested from the Cocus nucifera palm. The extracted oil has many uses, including applications in food, medicine and industry. It has a high level of saturated fat up to approximately >90% which sets it apart from other high saturated fats like palm oil. A typical fatty acid profile is seen in table 1. Coconut oil can be extracted in all standard methods of oil extraction including expeller pressed to solvent methods. In most cases coconut oil is RBD (refined, bleached and deodorized) following extraction.

In certain embodiments both coconut oil extracted by both heat and cold pressing can be used with this invention. It is reported that expeller pressing with heat can improve the level of antioxidants.

The preferred embodiment for this invention is to use “virgin or extra virgin” coconut oil with the various methods of application listed in this description. No other additives are needed for the preferred embodiment. Coconut oil is generally regarded as safe (GRAS) and we have exemplified that it has natural lubricant properties for vaginal atrophy not previously reported. The coconut oil has shown to have extraordinary properties that allow vaginal atrophy to be reduced and allow the vagina to maintain its natural properties even when estrogen is limiting.

Here we describe the method to apply coconut oil to the vagina. The difficulty with many lubricants is that they are messy and application is often at the time of sexual intercourse. Precise application is essential for the best outcome. Spillage or leakage is not acceptable and application prior to sexual intercourse is preferred. The methods described here clearly have the desired outcome of reducing vaginal atrophy while allowing the application to meet with acceptability.

Vaginal application of feminine products has been well documented and described over many years. In this case we have explored four main methods of application that include direct insertion of a tampon (with or without applicator), suppository/aliquot with or without applicator, vaginal sponge with or without applicator, or a douche packet.

In certain embodiments the application of the coconut oil to the vagina can be achieved by delivering approximately 2-18 mL of coconut oil to the vagina via a infused tampon. Tampons with a sealed, perforated end “applicator” system allows for the most acceptable non-messy insertion of the tampon to the vagina. Coconut oil infused tampons can be easily managed as the tampon is designed to hold liquid far beyond the volumes needed for this application. The modified applicator will hold an infused tampon in either liquid or solid form. Woman use this style of tampon application routinely worldwide.

In certain embodiments the application of the coconut oil to the vagina can be achieved by using solid coconut oil of approximately 2-8 mL (at a temperature below the melting point of the oil). Suppositories have been used to deliver many products for human health to enhance drug uptake or deliver more directly to areas of the body such as the anal canal. It is clear that this method allows the direct application of the coconut oil to reduce vaginal atrophy.

Coconut oil can be delivered to the vagina via a sponge infused with approximately 2-14 mL.

A Douche Packet can be easily inserted into the vagina (or anal canal) delivering 2-12 mL of coconut oil.

### BRIEF DESCRIPTION OF THE DRAWINGS

### Example 1

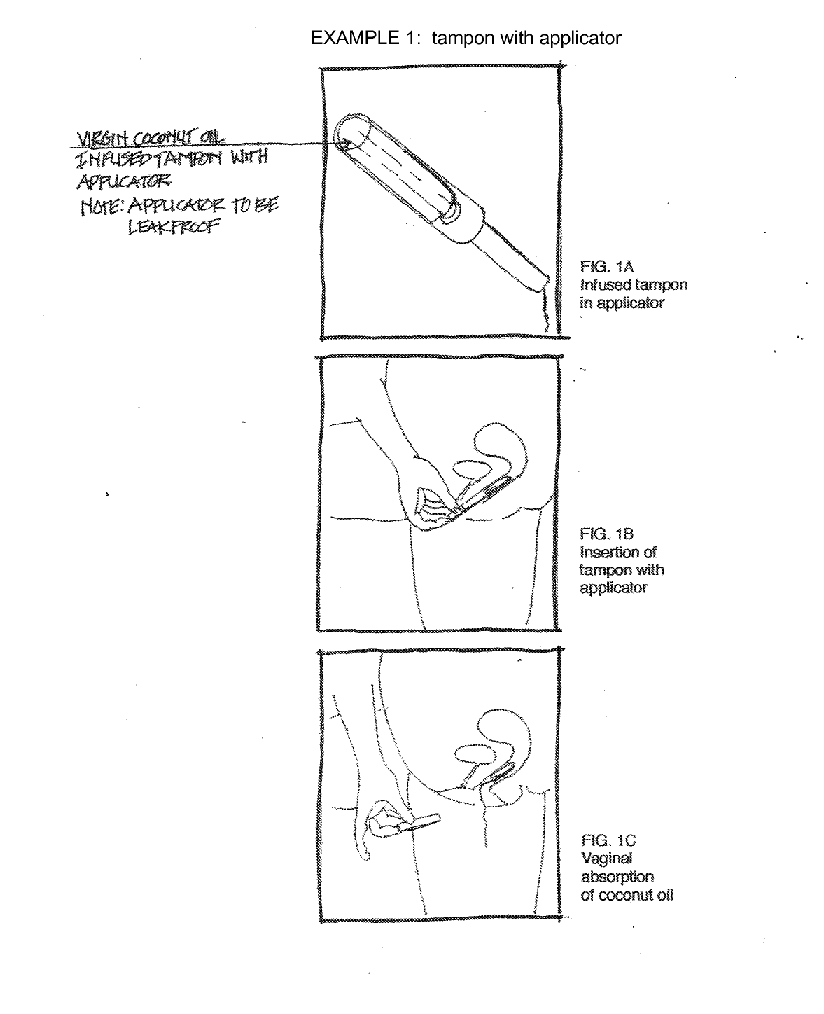

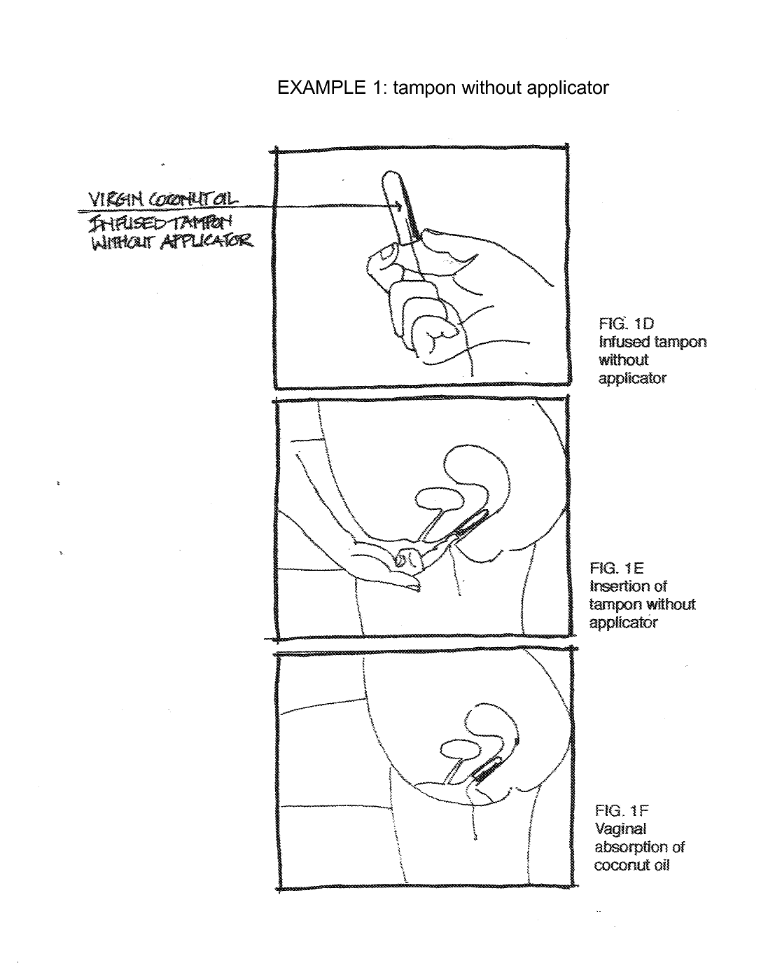

### FIGS. 1A, 1B, 1C, 1D, 1E, 1F

The coconut oil, containing no additives (extra virgin oil), in its liquid form (usually room temperature ˜22° C.) was added to tampons by an eyedropper. Tampons were either in the applicator provided with the tampons as bought or without the applicator.

Approximately 2-18 mL was easily added to the tampon without any tampon expansion, it was best achieved when the tampon was contained within an applicator.

Once the liquid was applied the tampon was inserted into the vagina as per the standard instructions either manually or with the provided applicator.

Vaginal atrophy associated pain with sexual penetration or intercourse was noticeably reduced within hours after one application. After one to five applications the atrophy was reduced for many days. These long-lasting effects were not anticipated.

### Example 2

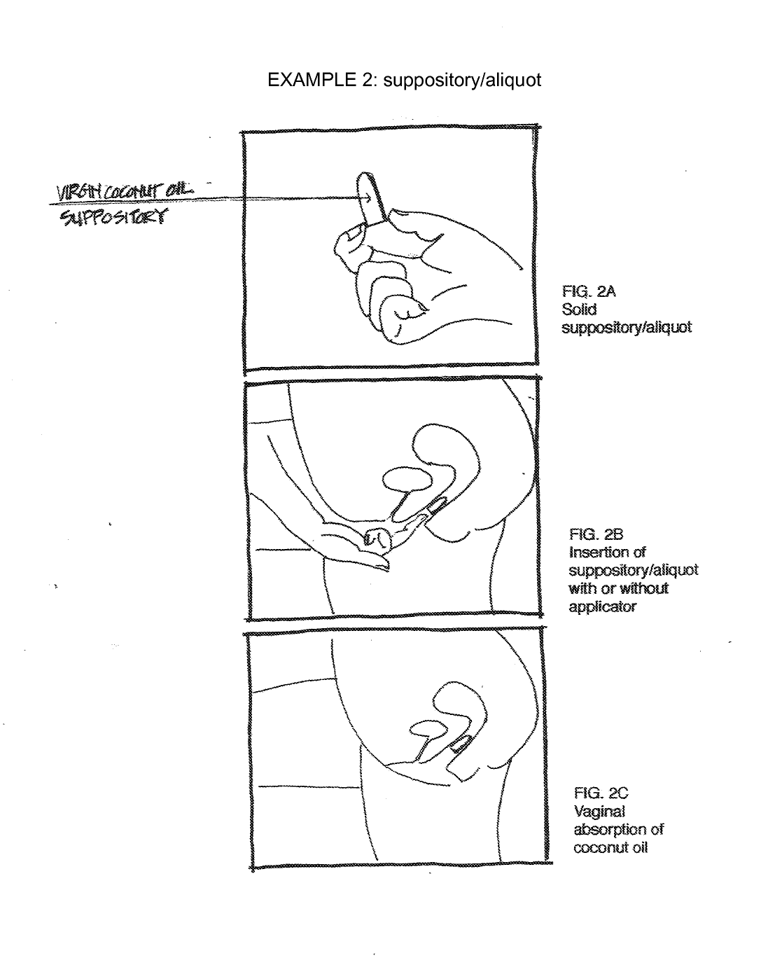

### FIGS. 2A, 2B, 2C

Coconut oil (extra virgin) was melted and divided into suppositories/aliquots of 2-8 mL and then either frozen, refrigerated or left at a temperature of less than 22^{C} to solidify.

Suppositories/aliquots were inserted into the vagina with or without applicator. The coconut oil melted on contact within the vagina.

Similar to example 1. Vaginal atrophy associated pain with sexual penetration or intercourse was noticeably reduced within hours after one application. After one to five applications the atrophy was reduced for many days. These long-lasting effects were not anticipated.

### Example 3

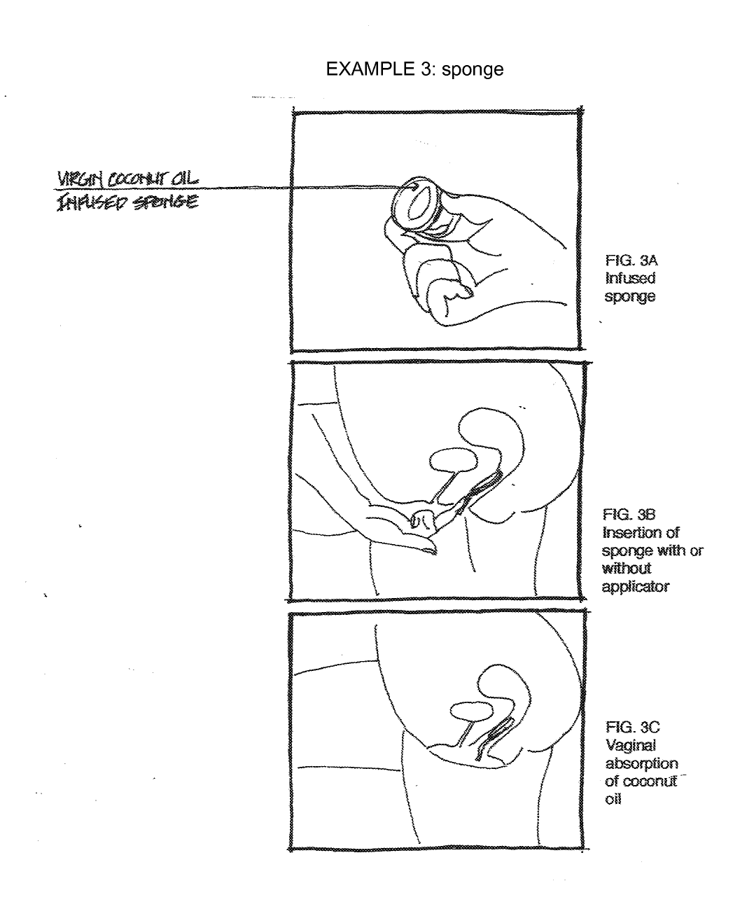

### FIGS. 3A, 3B, 3C

Approximately 2-18 mL of Coconut oil can be melted and added to a vaginal sponge.

The coconut oil can be applied to the sponge and then inserted into the vagina as per the standard instructions either manually with or without applicator.

It is predicted that Vaginal atrophy associated pain with sexual penetration or intercourse can be reduced within hours after one application. After one to five applications the atrophy could be reduced for many days.

### Example 4

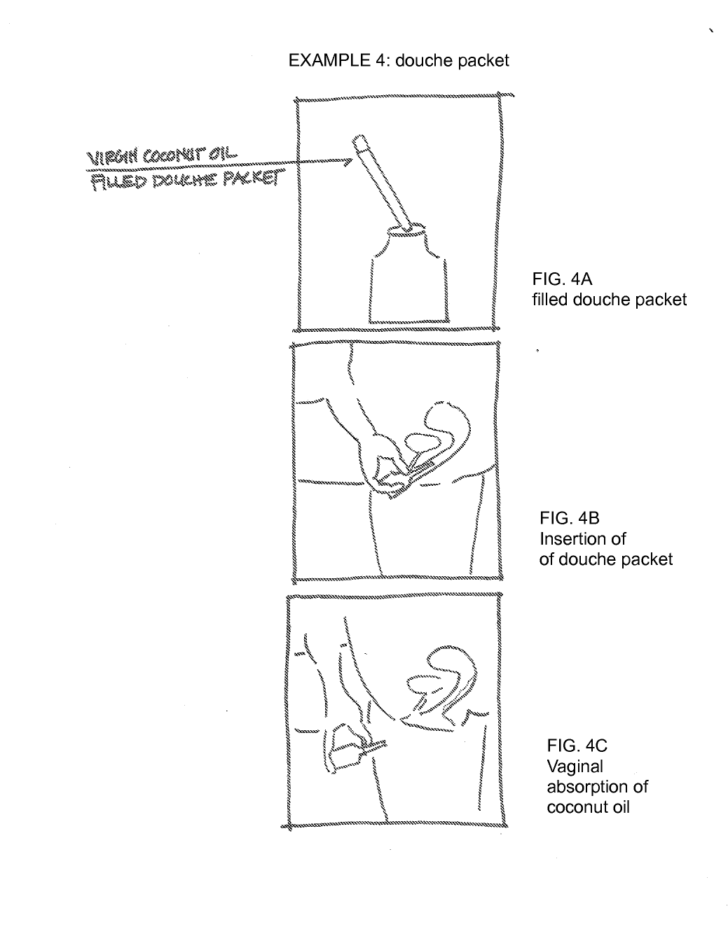

### FIGS. 4A, 4B, 4C

Approximately 2-18 mL of Coconut oil in a Douche Packet can be delivered to the vagina.

It is predicted that Vaginal atrophy associated pain with sexual penetration or intercourse can be reduced within hours after one application. After one to five applications the atrophy could be reduced for many days.

TABLE 1
A generalized fatty acid composition of Coconut Oil Name of Fatty Acid
Percentage (weight) %

| Fatty Acid | Weight Percent % |
|---|---|
| Lauric | 45-52 |
| Myristic | 16-21 |
| Caprylic | 5-10 |
| Capric | 4-8 |
| Caproic | 0.5-1 |
| Palmitic | 7-10 |
| Oleic | 5-8 |
| Palmitoleic | <1 |
| Linoleic | 1-3 |
| Linolenic | <1 |
| Stearic | 2-4 |

We claim:

1. A method of treating vaginal atrophy by the application or delivery of all natural coconut oil to the vagina.

2. A method of claim 1 where the delivery is done using a tampon infused with coconut oil.

3. A method of claim 1 where the delivery of the tampon infused with coconut oil is delivered to the vagina using a tampon applicator.

4. A method of claim 1 where the treatment reduces vaginal atrophy similar to that experienced during menopause.

5. A natural form of coconut oil expeller, heat or cold pressed from coconut fruit either in the form of refined or unrefined coconut oil that when delivered directly to a woman's vagina reduces the symptoms of vaginal atrophy.
